(19) United States
(12) Patent Application Publication
(10) Pub. No.: US 2018/0171093 A1
(43) Pub. Date: Jun. 21, 2018

(54) METALLIZED OPEN-CELL FOAMS AND FIBROUS SUBSTRATES

(71) Applicant: UNIVERSITÄT BAYREUTH, Bayreuth (DE)

(72) Inventors: Andreas Greiner, Bayreuth (DE); Seema Agarwal, Marburg (DE); Markus Langner, Bayreuth (DE)

(21) Appl. No.: 15/552,527

(22) PCT Filed: Feb. 25, 2016
(86) PCT No.: PCT/EP2016/054011

§ 371 Date: Aug. 22, 2017

(30) Foreign Application Priority Data
Feb. 25, 2015 (EP) 15156521.5

(51) Int. Cl. C08J 9/40; C08F 26/06; C08G 16/02; A61L 2/238; C08J 9/42; F21V 19/00

(52) U.S. Cl. CPC C08J 9/40; C08F 26/06; C08G 16/0268; C08G 2101/00; C08J 9/42; F21V 19/0025; A61L 2/238

(57) ABSTRACT

The present invention relates to a method for preparing a metallized open-cell foam or fibrous substrate, wherein the method comprises: (A) providing an open-cell foam or fibrous substrate, wherein the open-cell foam or fibrous substrate contains a polymer comprising heteroatom-containing moieties within the bulk of the open-cell foam or fibrous substrate or as a coating on the open-cell foam or fibrous substrate, wherein the polymer comprising heteroatom-containing moieties is selected from polyvinylpyridine, polyvinylpyrrolidone, polyvinyl alcohol, polyallylamine, polyethylene oxide, polyethylene imine, polyethylene sulfide and copolymers or blends thereof; (B) contacting the open-cell foam or fibrous substrate with nanoparticles of a first metal to provide a nanoparticle coated open-cell foam or fibrous substrate; and (C) contacting the nanoparticle coated open-cell foam or fibrous substrate with a solution comprising a salt of a second metal and a reducing agent to provide the metallized open-cell foam or fibrous substrate having a layer of the second metal on the nanoparticle coated open-cell foam or fibrous substrate.

### FIELD OF THE INVENTION

The present invention relates to metallized open-cell foams or metallized fibrous substrates, methods of preparing the same and their applications.

### BACKGROUND OF THE INVENTION

Open-cell foams and fibrous substrates provide a wide range of properties which make them of interest in many fields. Metals are also indispensible in technical applications. Open-cell, two- and three-dimensional metal structures are known but they have properties which are similar to those of the bulk metals. If metals and polymers are combined, the properties of the metal, such as the electric conductivity, can be combined with those of the polymer, such as its elasticity or formability.

Metallization of open-cell foams and fibrous substrates could give novel polymer/metal composites with highly interesting properties for a variety of applications, e.g. electrodes in fuel cells, membranes, filtration, thermal insulation, heating, ultralight weight constructions, and catalysis. Well-established metallization techniques for polymer materials include physical or chemical vapor deposition and galvanization (Eaves, D. Handbook of Polymer Foams 2004, Smither Rapra Press). These techniques are not suitable for homogeneous metallizations of polymer open-cell foams due to their complex three dimensional structure. A particular problem is the import of metal inside the pores of open-cell foams and fibrous substrates and the formation of continuous metal layers on the substrate. A method of choice could be wet metallization also assigned as electroless plating. There are numerous reports on wet metallization of polymer surfaces following different concepts. Major pathways for copper plating of polymers are based on the treatment with copper salts and formaldehyde (Nakahara, S.; Okinaka, Y. Ann. Rev. Mater. Sci. 1991, 21, 93). For example, wet chemical deposition of copper was achieved by treatment of an open cell polyurethane open-cell foam with CuSO₄ and formaldehyde under basic conditions using PdCl₂ as an activator (Tian, Q-h; Guo, X-y Trans. Nonferrous Met. Soc. China 2010, 153, s283). Plasmonic copper nanoparticle coatings were prepared on silanized glass slides following an elegant approach using also CuSO₄ and formaldehyde but sodium tetrachlorogold (Susman, M. D.; Feldman, Y., Vaskevich, A. Rubinstein, I. Chem. Mater. 2012, 24, 2501). A number of efforts were undertaken using ultrasound for fast electroless plating (Cobley, A. J.; Mason, T. J.; Saez, V. Trans. Inst. Metal Fin. 2011, 89, 303). However, ultrasound assisted electroless plating required careful adjustment of the ultrasound frequency in order to avoid pitting of the metal surface due to microjetting, which could be also a major problem in the absence of ultrasound due to evolving hydrogen bubbles (Park, Y. S.; Kim, M. H.; Kwon, S. C. Surf. Coat. Technol. 2002, 20, 245). The importance of the quality of the metal layer obtained by electroless plating on polymer substrates corresponds directly with their electrical properties. Broken and grainy metal layers or pitted surfaces will significantly reduce their electrical conductivity. However, under suitable plating conditions nickel electrodes were produced with polyurethane foams (Inazawa, S.; Hosoe, A.; Majima, M.; Nitta, K. Sei Techn. Rew. 2010, 71, 23). The polyurethane foams served as sacrificial template. As a result highly conductive nickel open-cell foams with high porosities of up to 98% and pore sizes of several hundred micrometers were obtained which are used as cathode current collectors in metal hydride batteries.

Open-cell polymer structures with a foam structure were prepared by a variety of different materials. For example, highly porous poly(styrene-co-acrylonitrile) (SAN) open-cell foams were obtained by foaming with supercritical CO₂ (Kyung-Nam, L; Lee, H.-J.; Kim, J.-H. Polym. Intern. 2000, 49, 712). A highly versatile and also technically realized approach to ultralight, highly porous open-cell foams were realized by microwave assisted melamine formaldehyde (MF) resin synthesis (Wang, D.; Zhang, X.; Luo, S.; Li, S. Adv. Mater. Phys. Chem. 2012, 2, 63). Under appropriate conditions depending on the use of emulsifiers sponges with apparent densities as low as 5.53 mg cm⁻³ with good compressive strength were obtained. Electroless plating of open-cell melamine formaldehyde foams with AgNO₃ and PdCl₂ as an activator yielded silver coated open-cell foams with good conductivity (σ=1.6*10³S m⁻¹) and good electronic shielding properties (Xu, Y; Li, Y.; Xu, W., Bao, J. J. Mater. Sci. Electron. 2015, 26, 1159).

A problem of the known metallization methods is that they often require complex or laborious procedures and/or are restricted to certain polymer surfaces if high quality metallization is desired. Metallization of open-cell melamine formaldehyde (MF) foams which provides electrically conductive open-cell melamine formaldehyde foams with a specific resistance R<50 Ωmm² m⁻¹ is not known.

WO 2004/056699 refers to nanoparticles and nanoscopic structures which may be obtained by: a) mixing a block copolymer with at least one low-molecular component, b) phase separation of the mixture to give a nanostructure, c) a subsequent treatment to improve the macroscopic orientation of the structure generated and d) leaching of at least one of the low molecular weight components such that nanoscopic holes or nanoparticles with a size from one to two hundred nm are produced.

It has been desired to provide novel materials which combine the desirable properties of open-cell foams or fibrous substrates with those of metals. In particular, it is desired to provide high quality metal layers on open-cell melamine formaldehyde foams and fibrous substrates.

### SUMMARY OF THE INVENTION

In a first aspect, the present invention refers to a method for preparing a metallized open-cell foam or fibrous substrate, wherein the method comprises:
(A) providing an open-cell foam or fibrous substrate, wherein the open-cell foam or fibrous substrate contains a polymer comprising heteroatom-containing moieties within the bulk of the open-cell foam or fibrous substrate or as a coating on the open-cell foam or fibrous substrate, wherein the polymer comprising heteroatom-containing moieties is selected from polyvinylpyridine, polyvinylpyrrolidone, polyvinyl alcohol, polyallylamine, polyethylene oxide, polyethylene imine, polyethylene sulfide and copolymers or blends thereof;
(B) contacting the open-cell foam or fibrous substrate with nanoparticles of a first metal to provide a nanoparticle coated open-cell foam or fibrous substrate; and
(C) contacting the nanoparticle coated open-cell foam or fibrous substrate with a solution comprising a salt of a second metal and a reducing agent to provide the metallized open-cell foam or fibrous substrate having a layer of the second metal on the nanoparticle coated open-cell foam or fibrous substrate.

In a second aspect, the present invention refers to a method for preparing a metallized open-cell foam or fibrous substrate, wherein the method comprises:
(i) providing an open-cell foam or fibrous substrate;
(ii) contacting the open-cell foam or fibrous substrate with a polymer comprising heteroatom-containing moieties, wherein the polymer comprising heteroatom-containing moieties is selected from polyvinylpyridine, polyvinylpyrrolidone, polyvinyl alcohol, polyallylamine, polyethylene oxide, polyethylene imine, polyethylene sulfide and copolymers or blends thereof, to provide a polymer coated open-cell foam or fibrous substrate;
(iii) contacting the polymer coated open-cell foam or fibrous substrate with nanoparticles of a first metal to provide a nanoparticle coated open-cell foam or fibrous substrate; and
(iv) contacting the nanoparticle coated open-cell foam or fibrous substrate with a solution comprising a salt of a second metal and a reducing agent to provide the metallized open-cell foam or fibrous substrate having a layer of the second metal on the nanoparticle coated open-cell foam or fibrous substrate.

In a third aspect, the present invention relates to a method for preparing a metallized open-cell foam or fibrous substrate, wherein the method comprises:
(a) providing an open-cell foam or fibrous substrate, wherein the open-cell foam or fibrous substrate comprises a polymer comprising heteroatom-containing moieties, wherein the polymer comprising heteroatom-containing moieties is selected from polyvinylpyridine, polyvinylpyrrolidone, polyvinyl alcohol, polyallylamine, polyethylene oxide, polyethylene imine, polyethylene sulfide and copolymers or blends thereof;
(b) contacting the open-cell foam or fibrous substrate with nanoparticles of a first metal to provide a nanoparticle coated open-cell foam or fibrous substrate; and
(c) contacting the nanoparticle coated open-cell foam or fibrous substrate with a solution comprising a salt of a second metal and a reducing agent to provide the metallized open-cell foam or fibrous substrate having a layer of the second metal on the nanoparticle coated open-cell foam or fibrous substrate.

Another aspect of the present invention relates to a metallized open-cell foam or fibrous substrate obtainable by the method according to the present invention.

A further aspect of the present invention relates to a lighting device comprising:

a first conductive layer comprising a first metallized open-cell foam or metallized fibrous substrate according to the present invention;

an insulating layer;

a second conductive layer, which preferably comprises a second metallized open-cell foam or metallized fibrous substrate according to the present invention; and

a lamp having a first electric contact which is connected to the first conductive layer and having a second electric contact which is connected to the second conductive layer.

In yet another aspect, the present invention relates to a heating device comprising a metallized open-cell foam or metallized fibrous substrate according to the present invention and at least two electric contacts.

The metallized open-cell foam or fibrous substrates according to the present invention can be used as a lighting device, a heating device, as a shield against electromagnetic irradiation, a filter, a catalyst, a substrate in analytical or preparative chromatography, or in a water separation device.

The metallized open-cell foam or fibrous substrates according to the present invention can also be used as a heat and/or sound insulation material, particularly in architectural applications, refrigeration devices or in vehicles.

### BRIEF DESCRIPTION OF THE FIGURES

FIG. 1. Scheme illustrating the metallization of an open-cell melamine formaldehyde foam (1) by deposition of P4VP (2), deposition of AgNP (3), and deposition of copper (4i) or silver (4ii).

FIG. 2. Increase in weight gain of open-cell foam (3) by metal uptake versus reaction time (A) and correlation of electrical conductivity with metal uptake (B).

FIG. 3. SEM micrographs of open-cell foam surfaces after 5 min (A), 10 min (B), 15 min (C), or 30 min (D) reaction time with AgNO₃ and after 5 min (E), 10 min (F), 15 min (G), or 30 min (H) reaction time with CuSO₄. Cross-sectional photograph of copper coated open-cell melamine formaldehyde foam after 30 min reaction time (I) and SEM micrograph after 120 min (K). Cross-sectional photograph of silver coated open-cell melamine formaldehyde foam after 210 min (J) and SEM micrograph after 120 min (L).

FIG. 4. Time-temperature plot of 4ii with and without current flow (A). Photograph and IR-camera image of 4ii at a wattage of 1W (B, C). Photograph of 1, 4ii, and ground 4ii on a heating plate at 100° C. (D) and corresponding IR-camera image (E).

FIG. 5. Photograph of a water droplet (left) and a paraffin droplet (right) on a silver coated open-cell melamine formaldehyde foam.

FIG. 6. Photograph of a water droplet (left) and a paraffin droplet (right) on a copper coated open-cell melamine formaldehyde foam.

FIG. 7. Agar plates with samples after 18 h incubation. Silver coated open-cell melamine formaldehyde foam vs. E. coli (left) and vs. M. luteus (right).

FIG. 8. Treatment with inoculation loop below the silver coated open-cell melamine formaldehyde foam vs. E. coli (18 h, left) and vs. M. luteus (48 h, right).

FIG. 9. Agar plates after incubation. Uncoated open-cell melamine formaldehyde foam vs. E. coli (18 h, left) and vs. M. luteus (48 h, right).

FIG. 10. Treatment with inoculation loop below the uncoated open-cell melamine formaldehyde foam vs. E. coli (18 h, left) and vs. M. luteus (48 h, right).

FIG. 11. Left: modified LED which can separately contact each silver coated open-cell melamine formaldehyde foam layer. Middle: Sandwich structure of two silver coated open-cell melamine formaldehyde foam layers and an uncoated open-cell melamine formaldehyde foam layer. Switched off. Right: Sandwich structure of two silver coated open-cell melamine formaldehyde foam layers and an uncoated open-cell melamine formaldehyde foam layer. Switched on.

FIG. 12. Thermograph and photograph of a open-cell melamine formaldehyde foam at a current of 5.2 A and a voltage of 0.195 V (1 W). The upper part of the open-cell melamine formaldehyde foam was silver coated while the lower part was uncoated.

FIG. 13. SEM image of the surface of a silver coated open-cell melamine formaldehyde foam without pretreatment with P4VP or AgNP with low (A, 500×) and high (B, 10 k×) magnification. Scale bar=50 μm and 2.5 μm.

FIG. 14. SEM image of the surface of a silver coated open-cell melamine formaldehyde foam with pretreatment with P4VP but without pretreatment with AgNP with low (A, 500×) and high (B, 10 k×) magnification. Scale bar=50 μm and 2.5 μm.

FIG. 15. SEM image of the surface of a copper coated open-cell melamine formaldehyde foam with pretreatment with P4VP and AgNP prepared in the presence of 2000 ppm PEG and in the absence of oxygen in reaction medium (A, 20 k×) and prepared in the presence of 200 ppm PEG in reaction medium under oxygen (B, 25 k×). Scale bar=1 μm.

FIG. 16. (A) TEM image of AgNP used for surface treatment of the P4VP coated open-cell melamine formaldehyde foam and measured size (B).

FIG. 17. Left: copper plated polyurethane foams. Right: untreated foam.

### DETAILED DESCRIPTION OF THE INVENTION

The present invention relates to methods for preparing a metallized open-cell foam or fibrous substrate.

The thickness of the layer of the second metal on the nanoparticle coated open-cell foam or fibrous substrate is not particularly limited and can, e.g., be about 50 nm to about 5 μm, more preferably 250 nm to about 2.5 μm.

### Second Embodiment of the Present Invention

Step (i): Providing an Open-Cell Foam or Fibrous Substrate

The open-cell foam or fibrous substrate can be any solid open-cell foam or fibrous substrate. In one embodiment, an open-cell foam is preferred.

Open-cell foams are foams which contain pores that are connected to each other and form a continuous interconnected network, so that gases and liquids can penetrate into the foam. This term is also intended to cover reticulated foams.

The porosity of the open-cell foam is not particularly limited and will depend on the desired end use. The porosity of the open-cell foam is preferably about 90 to about 99.9%. It can be measured, for instance, by porometer or by the following equation: Porosity=1−(bulk density/density of sample).

Open-cell foams can preferably have an air permeability of about 50 to about 15000 liter per square meter per second (l/(m²*s)) at a differential pressure of 200 Pa at 1 cm thickness.

Open-cell foams can preferably have a flow resistance of about 10 to about 15 kPa*s/m² as measured according to DIN EN ISO 7231.

The open-cell foams can preferably have a mass per unit area of about 5 g/m² to about 2000 g/m².

The open-cell foams can preferably have a density of about 1 kg/m³ to about 1000 kg/m³, more preferably about 1 kg/m³ to about 100 kg/m³.

The term “fibrous substrates” refers to any substrate which contains fibers, whereby the interstices between the fibers are not filled, so that gases and liquids can penetrate into the foam. This term covers nonwoven and woven materials.

Fibrous substrates can preferably have an air permeability of about 5 to about 5000 (l/(m²*s)) at a differential pressure of 200 Pa at 1 cm thickness

The fibrous substrates can preferably have a flow resistance of about 10 to about 15 kPa*s/m² as measured according to DIN EN ISO 7231.

The fibrous substrates can preferably have a mass per unit area of about 5 g/m² to about 1000 g/m²

The fibrous substrates can preferably have a density of about 1 kg/m³ to about 1000 kg/m³, more preferably about 1 kg/m³ to about 100 kg/m³.

The open-cell foam or fibrous substrate can comprise any material but typically comprises a polymer material. The polymer material is not particularly limited and can be selected from melamine formaldehyde resin, polyurethane, polyamide, polyimide, polyester polyurethane, polyether polyurethane, polyester, polyether, polyetherketone, phenol resin, polystyrene and combinations thereof. Preferably the polymer material is selected from melamine formaldehyde resin, polyurethane, polyamide, polyimide, polyester polyurethane, polyether polyurethane, and combinations thereof. More preferably the polymer material is selected from melamine formaldehyde resin, polyurethane, polyamide, polyimide, polyester polyurethane, polyether polyurethane, and combinations thereof. Even more preferably the polymer material is selected from melamine formaldehyde resin, polyurethane, polyester polyurethane, polyether polyurethane, and combinations thereof. Most preferably the polymer material is melamine formaldehyde resin.

A preferred open-cell foam is available from BASF SE under the trade designation Basotect®.

The air permeability can be measured, for instance, according to DIN EN ISO 9237.

Hereinafter the open-cell foam or fibrous substrate is sometimes collectively referred to as the “substrate”.

Step (ii) Contacting the Open-Cell Foam or Fibrous Substrate with a Polymer Comprising Heteroatom-Containing Moieties

The substrate is contacted with a polymer comprising heteroatom-containing moieties, wherein the polymer comprising heteroatom-containing moieties is selected from polyvinylpyridine, polyvinylpyrrolidone, polyvinyl alcohol, polyallylamine, polyethylene oxide, polyethylene imine, polyethylene sulfide and copolymers or blends thereof.

Although not wishing to be bound by theory, it is assumed that the polymer comprising heteroatom-containing moieties serves as a tie layer between the substrate and the nanoparticles of a first metal which are subsequently applied and thus serves to improve the adhesion of the nanoparticles of a first metal to the substrate.

Preferably, the polymer comprising heteroatom-containing moieties includes polyvinylpyridine (including poly(4-vinylpyridine) and poly(2-vinylpyridine)), polyvinylpyrrolidone, polyvinyl alcohol, polyvinyl thiol, or polyallylamine. A preferred polymer comprising heteroatom-containing moieties is polyvinylpyridine, polyethyleneimine or a copolymer thereof, wherein the copolymer includes at least about 0.1 wt.-% monomers derived from vinylpyridine or ethyleneimine, more preferably at least about 0.5 wt.-% monomers derived from vinylpyridine or ethyleneimine, even more preferably at least about 1 wt.-% monomers derived from vinylpyridine or ethyleneimine.

It is understood that the term “polymer comprising heteroatom-containing moieties” covers homopolymers and copolymers of the indicated monomers. For instance, “polyvinylpyridine” is intended to cover homopolymers of polyvinylpyridine as well as copolymers of vinylpyridine and at least one copolymerizable monomer. Due to the high affinity of the heteroatom-containing moieties to the first metal which is contained in the nanoparticles, the advantageous effects of the present invention can already be achieved with relatively low amounts of the monomers providing the heteroatom-containing moieties. For instance, the copolymer can include at least about 0.1 wt.-% monomers providing the heteroatom-containing moieties, more preferably at least about 0.5 wt.-% monomers providing the heteroatom-containing moieties, even more preferably at least about 1 wt.-% monomers providing the heteroatom-containing moieties.

Blends of the above mentioned polymer comprising heteroatom-containing moieties can also be used.

The weight average molecular weight of the polymer comprising heteroatom-containing moieties can preferably range from about 50 g/mol to about 5×10⁶ g/mol, more preferably about 10³ g/mol to about 10⁶ g/mol (as determined using a polystyrene standard).

The polymer comprising heteroatom-containing moieties can be contacted with the substrate in any desired manner. Methods for contacting the polymer comprising heteroatom-containing moieties and the substrate are well-known in the art and include impregnating, dipping, spraying, vapor deposition, and the like. Typically the polymer comprising heteroatom-containing moieties is provided as a solution, dispersion or emulsion and is then contacted with the substrate, e.g., by impregnating or dipping. The method of contacting will depend on the thickness and porosity of the substrate and how deep the polymer comprising heteroatom-containing moieties should be applied to the substrate. The polymer comprising heteroatom-containing moieties can be contacted with the open-cell foam or fibrous substrate either only to part of its thickness (which means a penetration depth of e.g., at least 5% of the thickness, at least 10% of the thickness, at least 20% of the thickness, at least 30% of the thickness, at least 40% of the thickness, at least 50% of the thickness, at least 60% of the thickness, at least 70% of the thickness, at least 80% of the thickness, or at least 90% of the thickness or can contact the complete thickness of the open-cell foam or fibrous substrate (i.e., a penetration depth of 100%). If a complete coating is desired, then methods such as impregnating and dipping are preferred. If a partial coating or a gradient coating is desired, then methods such as spraying and vapor deposition are preferred. In the present invention it is preferred to contact the complete thickness of the substrate, e.g., by impregnating or dipping.

The concentration of the polymer comprising heteroatom-containing moieties in the solution, dispersion or emulsion can be in the range of about 1 mg/l to about 200 g/l, preferably about 100 mg/l to about 20 g/l.

The solution, dispersion or emulsion comprises a liquid carrier or solvent in addition to the polymer comprising heteroatom-containing moieties. The liquid carrier or solvent in the solution, dispersion or emulsion is not particularly limited and will depend on the substrate and the polymer comprising heteroatom-containing moieties which are employed. The liquid carrier or solvent should not detrimentally affect or dissolve the substrate and should be able to adequately apply the polymer comprising heteroatom-containing moieties to the substrate. Furthermore, it should be easy to remove from the substrate after the contacting step. Examples of typical liquid carriers or solvents include water, C₁₋₄ alcohols (such as methanol, ethanol, 2-propanol, 1-propanol), THF, diethylether, toluene, water, acetone and mixtures thereof, preferably water, C₁₋₄ alcohols (such as methanol, and ethanol) and mixtures thereof.

The contacting step (ii) can be conducted under any suitable conditions. These can be chosen by a person skilled in the art based on the contacting method chosen.

If dipping or impregnation are employed, typically the temperature in the contacting step will range from about 0° C. to about 100° C., more preferably about 10° C. to about 30° C. The duration of the contacting step can be from about 1 s to about 1 h, preferably about 30 s to about 10 min. If necessary, pressure or underpressure can be applied.

If the polymer comprising heteroatom-containing moieties is applied in the form of a solution, dispersion or emulsion, the carrier liquid or solvent can be removed before the polymer coated substrate is contacted with the nanoparticles. Typical conditions for removing the carrier liquid or solvent depend on the carrier liquid and the solvent and include applying underpressure, increasing the temperature and combinations thereof.

The amount of the polymer comprising heteroatom-containing moieties which is applied to the substrate will depend on the final use and can be chosen appropriately by a skilled person.

Typically, the thickness of the layer of polymer comprising heteroatom-containing moieties which is applied to the fibers or cells of the substrate is about 0.1 nm to about 10000 nm, more preferably about 0.1 nm to about 1000 nm, even more preferably about 0.1 nm to about 100 nm, and even more preferably about 0.1 nm to about 10 nm. In further preferred embodiments, the thickness of the layer of polymer comprising heteroatom-containing moieties which is applied to the fibers or cells of the substrate is even more preferably about 1 nm to about 1000 nm, even more preferably about 1 nm to about 100 nm, even more preferably about 1 nm to about 30 nm, and even more preferably about 1 nm to about 10 nm. The thickness of the layer can be, e.g., determined by gravimetrical calculations, SEM, or TEM.

The polymer coated substrate should still be permeable after application of the polymer comprising heteroatom-containing moieties, so that the nanoparticles of a first metal and the solution comprising a salt of a second metal can enter into the bulk of the substrate.

The upper limit of the difference of the air permeability of the polymer coated substrate and the air permeability of the uncoated substrate is preferably less than about 25%, more preferably less than about 10%, even more preferably less than 5%. The lower limit of the difference of the air permeability of the polymer coated substrate and the air permeability of the uncoated substrate is typically more than about 0%, more typically more than about 1%.

Step (iii) Contacting the Polymer Coated Open-Cell Foam or Fibrous Substrate with Nanoparticles of a First Metal

The polymer coated substrate is subsequently contacted with nanoparticles of a first metal. The nanoparticles can be provided in a dispersion or they can be provided on the surface of a carrier.

The first metal is not particularly limited and can be any metal or metal alloy. Typical examples include transition metals and in particular Ag, Au, Pd, Pt, Rh, Ru, Cu, Ni, Ir and Os as well as alloys thereof. Preferred examples include Ag, Au, Pd, Pt, Rh, and Ru as well as alloys thereof, more preferably Ag and Au as well as alloys thereof.

Nanoparticles can be prepared from the first metal by methods which are known in the art.

Small nanoparticles (up to 10 nm) can be prepared from the respective metal salts and strong reducing agents, which are able to quickly reduce the metal ions to the respective elemental metal. Typical reducing agents include borohydrides (including NaBH₄, LiBH₄, lithium triethylborohydride and dimethylaminoborane) or hydrazine. Preferably stabilizers are added which reduce the aggregation of the nanoparticles. The concentration of the metal salt is also usually quite low.

If larger nanoparticles having a size of more than 10 nm are to be prepared, weaker reducing agents such as citrate, sugars (including glucose) aldehydes, EDTA, hydroquinone, reducing phenols or ascorbate can be employed. The concentrations of the metal salt and the reducing agent can be higher.

Examples of suitable methods are summarized in M.-C. Daniel et al., Chem. Rev., 2004, 104, 293-346. An exemplary method is described in C. Liu, Anal. Bioanal. Chem., 2011, 401, 229-235.

In a preferred method, nanoparticles are prepared by providing a solution of a salt of the first metal in a carrier liquid. The concentration of the salt of the first metal is generally in the range of about 0.1 mmol/l to about 100 mmol/l, preferably about 1 mmol/l to about 100 mmol/l. If desired, a stabilizer such as those listed below can be present, e.g., in a concentration of about 0.1 mmol/l to about 100 mmol/l, preferably about 1 mmol/l to about 100 mmol/l. Then a reducing agent is added. The reducing agent is not particularly limited and can be selected from borohydrides (including NaBH₄, LiBH₄, lithium triethylborohydride and dimethylaminoborane). The concentration of the reducing agent can be in the range of about 0.1 mmol/l to about 100 mmol/l, preferably about 1 mmol/l to about 100 mmol/l. The reduction can be carried out at any suitable temperature such as about 0° C. to about 90° C. It is desirable to vigourously agitate the solution and to add the reducing agent quickly.

The nanoparticles of the first metal typically have a size of about 1 nm to about 1000 nm, preferably about 1 nm to about 100 nm, more preferably about 4 nm to about 10 nm. The size of the nanoparticles can be determined by SEM or TEM.

Typically the nanoparticles of the first metal will be provided in the form of a dispersion which comprises a carrier liquid and the nanoparticles of the first metal. The liquid carrier in the dispersion is not particularly limited and will depend on the substrate, the polymer comprising heteroatom-containing moieties and the nanoparticles of a first metal which are employed. The liquid carrier or solvent should not detrimentally affect or dissolve the substrate or the polymer comprising heteroatom-containing moieties and should be able to adequately apply the nanoparticles to the polymer coated substrate. Furthermore, it should be easy to remove from the substrate after the contacting step. Examples of typical liquid carriers include organic solvents and water. Examples of liquid carriers include C₁₋₄ alcohols, halogenated organic solvents (including carbon tetrachloride, chloroform, dichloromethane, and chloromethane), ether solvents (including diethylether and THF), amide solvents (including dimethylformamide) and hydrocarbon solvents (including hexane). Preferred examples include water, C₁₋₄ alcohols and mixtures thereof, more preferably water. If desired, the dispersion can also comprise a stabilizer in order to stablize the dispersion. Examples of suitable stabilizers include, but are not limited to, citrate, sodium dodecylsulfonate, polymers containing heteroatoms (such as N, O and S), thiols, alcohols, amines, and phosphines.

The nanoparticles of the first metal are contacted with the polymer coated substrate by any suitable method. Methods for contacting the polymer coated substrate and the nanoparticles of the first metal are well-known in the art and include impregnating, dipping, and spraying, preferably impregnating or dipping. The method of contacting will depend on the thickness and porosity of the substrate and how deep the nanoparticles of the first metal should be applied to the substrate. The nanoparticles of the first metal can be contacted with the open-cell foam or fibrous substrate either only to part of its thickness (e.g., at least 5% of the thickness, at least 10% of the thickness, at least 20% of the thickness, at least 30% of the thickness, at least 40% of the thickness, at least 50% of the thickness, at least 60% of the thickness, at least 70% of the thickness, at least 80% of the thickness, or at least 90% of the thickness) or can contact the complete thickness of the open-cell foam or fibrous substrate. If a complete coating is desired, then methods such as impregnating and dipping are preferred. If a partial coating or a gradient coating is desired, then methods such as spraying are preferred. In the present invention it is preferred to completely coat the substrate, e.g., by impregnating or dipping.

The concentration of the nanoparticles of the first metal in the dispersion can be in the range of about 1 mg/l to about 200 g/l, preferably about 1 mg/l to about 20 g/l.

The contacting step (iii) can be conducted under any suitable conditions. These can be chosen by a person skilled in the art based on the contacting method chosen.

If dipping or impregnation are employed, typically the temperature in the contacting step will range from more than 0° C. to about 90° C., more preferably about 20° C. to about 40° C. The duration of the contacting step can be from about 1 s to about 10 days, preferably about 1 hour to about 4 days. If necessary, pressure or underpressure can be applied.

If desired, step (iii) can be repeated several times until the desired loading of the nanoparticles of the first metal has been achieved.

If the nanoparticles of a first metal are applied in the form of a dispersion, the carrier liquid can be removed before the nanoparticle coated substrate is contacted with the solution comprising a salt of a second metal and a reducing agent. Typical conditions for removing the carrier liquid depend on the carrier liquid and include applying underpressure, increasing the temperature and combinations thereof.

The amount of the nanoparticles which are applied to the substrate will depend on the final use and can be chosen appropriately by a skilled person.

Typically, the amount of the nanoparticles which is applied to the substrate is about 0.01 wt. % to about 20 wt.-%, more preferably about 1 wt.-% to about 10 wt.-%. The amount can be, e.g., determined by gravimetry or thermogravimetry (TGA).

The nanoparticle coated substrate should still be permeable after application of the nanoparticles of a first metal, so that the solution comprising a salt of a second metal can enter into the bulk of the substrate.

Without wishing to be bound by theory, it is assumed that the first metal can bind or associate with the heteroatoms of the polymer comprising heteroatom-containing moieties and thus be attached to the substrate.

Step (iv) Contacting the Nanoparticle Coated Open-Cell Foam or Fibrous Substrate with a Solution Comprising a Salt of a Second Metal and a Reducing Agent

The nanoparticle coated substrate is contacted with a solution comprising a salt of a second metal and a reducing agent to provide the metallized open-cell foam or fibrous substrate.

The second metal is not particularly limited and can be any metal or metal alloy. The second metal can be the same or different than the first metal. Typical examples include Ag, Au, Pd, Pt, Rh, Ru, Cu, Ir and Os as well as alloys thereof. Preferred examples include Ag, Cu, Au, Pd, Pt, Rh, and Ru as well as alloys thereof, more preferably Ag, Cu and Au as well as alloys thereof.

The salt of the second metal is reduced to elemental second metal by the reducing agent. This reaction is commonly termed “electroless plating” and is known in the art. Details on the reducing agents and conditions can be found, e.g., in “Electroless Plating—Fundamentals and Applications”, G. O. Mallory; J. B. Hajdu; William Andrew Publishing, 1990 (in particular “12. Fundamental Aspects of Electroless Copper Plating” and Electroless Plating of Silver) and in “Modern Electroplating”, 5^{1h} edition, 2010, John_Wiley & Sons, Inc. (in particular “5. Electroless and electrodeposition of silver” and “17. Electroless deposition of copper”). The contents of these references are incorporated herein by reference.

The reducing agent can be any reducing agent which reduces the salt of the second metal to the second metal and thus to form a layer of the second metal on the nanoparticle coated open-cell foam or fibrous substrate. Such reducing agents are well-known in the art of electroless plating and can be chosen by a skilled person according to the second metal which is to be reduced.

Examples of reducing agents include formaldehyde, glyoxal, aldehydes, dimethylamine borane, hydrazine, borohydrides, hypophosphites, sugars (e.g., glucose), Rochelle salt, Sn²⁺, and Fe²⁺. Preferred reducing agents include sugars and formaldehyde.

Silver can, for example, be reduced using a sugar such as glucose as a reducing agent. Copper can, for example, be reduced using, for instance, formaldehyde, dimethylaminoborane, or hypophosphite as a reducing agent.

The salt of the second metal and the reducing agent can be provided in a solution.

The salt of the second metal and the reducing agent can be contacted with the nanoparticle coated substrate in any desired manner. Methods for contacting the salt of the second metal and the reducing agent and the nanoparticle coated substrate are well-known in the art and include impregnating, dipping, spraying, and the like. Typically the salt of the second metal and the reducing agent are provided as a solution and are then contacted with the substrate, e.g., by impregnating or dipping. The method of contacting will depend on the thickness and porosity of the substrate and how deep the salt of the second metal and the reducing agent should be applied to the substrate. The salt of the second metal and the reducing agent can be contacted with the open-cell foam or fibrous substrate either only to part of its thickness (e.g., at least 5% of the thickness, at least 10% of the thickness, at least 20% of the thickness, at least 30% of the thickness, at least 40% of the thickness, at least 50% of the thickness, at least 60% of the thickness, at least 70% of the thickness, at least 80% of the thickness, or at least 90% of the thickness) or can contact the complete thickness of the open-cell foam or fibrous substrate. If a complete coating is desired, then methods such as impregnating and dipping are preferred. If a partial coating or a gradient coating is desired, then methods such as spraying are preferred. In the present invention it is preferred to completely coat the substrate, e.g., by impregnating or dipping.

The concentration of the salt of the second metal in the solution can be in the range of about 100 mg/l to about 200 g/l, preferably about 5 g/l to about 50 g/l.

The concentration of the reducing agent in the solution can be in the range of about 100 mg/l to about 200 g/l, preferably about 5 g/l to about 50 g/l.

The solution comprises a solvent in addition to the salt of the second metal and the reducing agent. The solvent is not particularly limited and will depend on the components which are employed. The solvent should not detrimentally affect or dissolve the substrate, the polymer comprising heteroatom-containing moieties, or the nanoparticles and should be able to adequately contact the salt of the second metal and the reducing agent with the nanoparticle coated substrate. Furthermore, it should be easy to remove from the substrate after the contacting step. Examples of typical solvents include water.

The contacting step (iv) can be conducted under any suitable conditions. These can be chosen by a person skilled in the art based on the contacting method chosen.

If dipping or impregnation are employed, typically the temperature in the contacting step will range from more than about 0° C. to about 90° C., more preferably about 30° C. to about 70° C. The duration of the contacting step can be from about 30 s to about 4 days, preferably about 30 min to about 5 h. If necessary, pressure or underpressure can be applied.

The quality of the metal layer can be improved, if the reduction of the second metal salt is conducted slowly. This allows gas bubbles, e.g., of hydrogen, to exit from the substrate and thus to improve the contact of the solution comprising the salt of the second metal and the reducing agent with the substrate. One option to reduce the reaction rate of the reduction of the second metal salt is to incorporate a complexing agent into the solution. The complexing agent is not particularly limited and can be any complexing agent which can complex the metal ion of the second metal salt. Typical examples of the complexing agent include multivalent carboxylic acids (including tartaric acid, oxalic acid, ascorbic acid, citric acid, and EDTA), cyanides and other ligands. Preferred complexing agents include tartaric acid and EDTA. The complexing agents are typically employed in stochiometric amounts or in excess compared to the number of moles of the metal cation of the second metal salt.

Another approach to improving the quality of the metal layer is to incorporate a surface active agent in the solution comprising the salt of the second metal and the reducing agent. The surface active agent is not particularly limited but includes nonionic and ionic surface active agents, preferably nonionic surface active agents. Examples of suitable surface active agents include polysorbate surface active agents (including those available under the trade designation “Tween”), poloxamer surface active agents (including those available under the trade designation “Pluronic”), polyethylene oxides and polypropylene oxides, preferably polyethylene oxides and polypropylene oxides. The amount of the surface active agent will depend on various factors including its specific type. Generally, the amount of the surface active agent can be in the range of about 1 mol-% to about 500 mol-% based on the number of moles of the metal cation of the second metal salt.

The solvent should be removed after the substrate has been contacted with the solution comprising a salt of a second metal and a reducing agent. Typical conditions for removing the carrier liquid depend on the carrier liquid and include applying underpressure, increasing the temperature and combinations thereof.

The amount of the second metal which is applied to the substrate will depend on the final use and can be chosen appropriately by a skilled person.

Typically, the amount of the second metal which is applied to the substrate is about 0.1 wt.-% to about 30 wt.-%, more preferably about 20 wt.-% to about 70 wt.-%. The amount can be, e.g., determined by gravimetrically or thermogravimetrically (TGA) or via ICP-OES.

The metallized open-cell foam or fibrous substrate should still be permeable after application of the second metal.

Without wishing to be bound by theory, it is assumed that the nanoparticles of the first metal act as “seeds” for the deposition of the second metal on the substrate. This enables a continuous layer of the second metal to be formed on the substrate. If the nanoparticles of the first metal are not employed, then only a discontinuous layer of the second metal with many defects or individual particles of the second metal are formed.

### Third Embodiment of the Present Invention

If the open-cell foam or the fibrous substrate comprise a polymer comprising heteroatom-containing moieties, wherein the heteroatoms are selected from N, O and S, then step (ii) can be omitted and the open-cell foam or the fibrous substrate can be directly contacted with the nanoparticles of a first metal. In this embodiment, the method comprises:
(a) providing an open-cell foam or fibrous substrate, wherein the open-cell foam or fibrous substrate comprises a polymer comprising heteroatom-containing moieties, wherein the heteroatoms are selected from N, O and S;
(b) contacting the open-cell foam or fibrous substrate with nanoparticles of a first metal to provide a nanoparticle coated open-cell foam or fibrous substrate; and
(c) contacting the nanoparticle coated open-cell foam or fibrous substrate with a solution comprising a salt of a second metal and a reducing agent to provide the metallized open-cell foam or fibrous substrate having a layer of the second metal on the nanoparticle coated open-cell foam or fibrous substrate.

The amount of the polymer comprising heteroatom-containing moieties in the open-cell foam or the fibrous substrate is not particularly limited and can be any suitable amount which allows the deposition of the nanoparticles of the first metal. It can be, for example, at least about 20 wt.-%, at least about 30 wt.-%, at least about 40 wt.-%, at least about 50 wt.-%, at least about 60 wt.-%, at least about 70 wt.-%, at least about 80 wt.-%, at least about 90 wt.-% or 100 wt.-%, based on the total weight of the open-cell foam or the fibrous substrate.

In the third embodiment, the polymer comprising heteroatom-containing moieties is preferably present throughout the bulk of the open-cell foam or fibrous substrate and is not just present as a coating on the surface.

The description of the steps given above with the second embodiment applies to corresponding steps of the third embodiment.

Applications

The metallized open-cell foam or fibrous substrate can be employed in a wide variety of fields. The metallized open-cell foams or fibrous substrates combine the desirable features (in particular the air permeability) of the open-cell foam or fibrous substrate with those of the second metal. Therefore, the present invention can provide lightweight electrically conducting open-cell foams and fibrous substrates which have good mechanical properties without detrimentally affecting the permeability of the open-cell foam or fibrous substrate.

A possible application of the metallized substrates is in the lighting field. In particular, the present application provides a lighting device comprising:

a first conductive layer comprising a first metallized open-cell foam or metallized fibrous substrate according to the present invention;

a second conductive layer, which preferably comprises a second metallized open-cell foam or metallized fibrous substrate according to the present invention;

an insulating layer between the first conductive layer and the second conductive layer; and a lamp (such as a light-emitting diode (LED) or a light bulb), having a first electric contact which is connected to the first conductive layer and having a second electric contact which is connected to the second conductive layer.

Because the first conductive layer comprises the first metallized substrate according to the present invention, which is metallized in the bulk, it is possible to easily position and reposition the lamp at any position of the first conductive layer. This allows for a completely new lighting concept in which the positions of the lamps and their number can be adapted according to the needs and/or individual design ideas of the customers.

The metallized substrates of the present application can also be used in a heating device. The heating device comprises a metallized open-cell foam or metallized fibrous substrate according to the present invention and at least two electric contacts or terminals electrically connected with the metallized open-cell foam or metallized fibrous substrate. The electric contacts or terminals are preferably provided at opposite sides of metallized open-cell foam or metallized fibrous substrate. Alternatively, the electric contacts or terminals are provided on the same side of the metallized open-cell foam or metallized fibrous substrate although being spaced from each other.

When electricity is passed from one terminal through the metallized substrate to the second terminal, it heats up and emits the heat within a very short amount of time. When the current flow is stopped, the heat emission stops within a very short amount of time. Importantly, the heat insulation properties of the underlying substrate are not detrimentally affected by the metallization, so that the heating device combines the features of heat emission and heat insulation. By a suitable combination of metallized and non-metallized substrates directional heat emission can be achieved.

It is also envisaged that the instant metallized substrates can be used as a heat and/or sound insulation material, particularly in architectural applications, refrigeration devices or in vehicles. The instant metallized substrates are particularly suitable for these applications because they retain the desired mechanical properties of the employed substrate. In contrast to the uncoated substrate which typically regains its initial shape after compression, the metallized substrates can be formed into a desired form and retain this shape. This is of particular interest for forming shaped parts which have to have to specific dimensions. Due to their hydrophobicity and antibacterial properties the metallized substrates can reduce mold formation. Furthermore, their properties as lighting devices and heating devices can also be of interest in these fields.

It is also envisaged that the instant metallized substrates can be used as a shield against electromagnetic irradiation because it has a good shielding efficiency against electrical and magnetic fields while its permeability allows air exchange. Due to the conductive properties of the metallized substrate they can also be used as a breathable, conductive, thermally isolating and soft shielding agent against electromagnetic waves. The shielding is therefore defined as the reduction of the effect of electric or magnetic fields from one side of the source of the fields through the material to the target that has to be protected on the other side of the material. The shielding can also be used to protect two circuits from influencing each other (S. Selozzi, R. Araneo, G. Lovat, Electromagnetic Shielding, Wiley & Sons, Inc., Hoboken, N.J., 2008). The properties like thermal insulating behavior and breathability are of interest in applications where electromagnetic shielding and these properties are useful.

It is also envisaged that the instant metallized substrates can be used as antistatic filters, in particular as active filters. Due to their permeability they can be used in filtration applications. The metal layer on the substrate can be used, e.g., to remove biological (e.g., proteins, enzymes, amino acids, viruses, bacteria or cells) or chemical (e.g., organic compounds, complex ligands, complexes, monomers, gases, or solvents) contaminants by chemical reaction or adsorption.

It is also envisaged that the instant metallized substrates can be used as catalysts. Due to their permeability they can be used as solid catalysts, for example, in gas phase or liquid phase catalytic reactions. In this application, the metal layer on the substrate is used as the catalyst.

It is also envisaged that the instant metallized substrates can be used as a substrate in analytical or preparative chromatography. For instance, liquid or organic substances could be selectively adsorbed on the instant metallized substrates.

It is also envisaged that the instant metallized substrates can be used for water separation The instant metallized substrates are particularly hydrophobic and oleophilic. Consequently, they can be employed to separate, e.g., mixtures of oil and water. A possible application is the separation of water from fuel (e.g., water from kerosene, gasoline or diesel) or fuel from water.

It is also envisaged that the instant metallized substrates can be used in a water management concept. In one application of this embodiment, liquid water can be injected into a cavity in an instant metallized substrate. Water which is present in liquid form remains in the cavity, while water which is in the form of vapor can diffuse from the substrate. In this manner, liquid water can be incorporated into the substrate without the periphery of the substrate being wetted. Consequently, the instant metallized substrates can be used as a leak proof moisture reservoir which is permeable and thus can provide a novel type of moisture control. This concept can also be used to provide water conduits by forming conduits in the metallized substrate through which water can flow.

The invention is summarized in the following items:
1. Method for preparing a metallized open-cell foam or fibrous substrate, wherein the method comprises:
   (A) providing an open-cell foam or fibrous substrate, wherein the open-cell foam or fibrous substrate contains a polymer comprising heteroatom-containing moieties within the bulk of the open-cell foam or fibrous substrate or as a coating on the open-cell foam or fibrous substrate, wherein the polymer comprising heteroatom-containing moieties is selected from polyvinylpyridine, polyvinylpyrrolidone, polyvinyl alcohol, polyallylamine, polyethylene oxide, polyethylene imine, polyethylene sulfide and copolymers or blends thereof;
   (B) contacting the open-cell foam or fibrous substrate with nanoparticles of a first metal to provide a nanoparticle coated open-cell foam or fibrous substrate; and
   (C) contacting the nanoparticle coated open-cell foam or fibrous substrate with a solution comprising a salt of a second metal and a reducing agent to provide the metallized open-cell foam or fibrous substrate having a layer of the second metal on the nanoparticle coated open-cell foam or fibrous substrate.
2. Method for preparing a metallized open-cell foam or fibrous substrate, wherein the method comprises:
   (i) providing an open-cell foam or fibrous substrate;
   (ii) contacting the open-cell foam or fibrous substrate with a polymer comprising heteroatom-containing moieties, wherein the polymer comprising heteroatom-containing moieties is selected from polyvinylpyridine, polyvinylpyrrolidone, polyvinyl alcohol, polyallylamine, polyethylene oxide, polyethylene imine, polyethylene sulfide and copolymers or blends thereof, to provide a polymer coated open-cell foam or fibrous substrate;
   (iii) contacting the polymer coated open-cell foam or fibrous substrate with nanoparticles of a first metal to provide a nanoparticle coated open-cell foam or fibrous substrate; and
   (iv) contacting the nanoparticle coated open-cell foam or fibrous substrate with a solution comprising a salt of a second metal and a reducing agent to provide the metallized open-cell foam or fibrous substrate having a layer of the second metal on the nanoparticle coated open-cell foam or fibrous substrate.
3. The method according to item 1 or 2, wherein the open-cell foam or fibrous substrate comprises polymer material which is selected from melamine formaldehyde resin, polyurethane, polyamide, polyimide, polyester polyurethane, polyether polyurethane, and combinations thereof, preferably wherein the open-cell foam or fibrous substrate comprises melamine formaldehyde.
4. The method according to any of items 1 to 3, wherein the polymer comprising heteroatom-containing moieties comprises polyvinylpyridine.
5. Method for preparing a metallized open-cell foam or fibrous substrate, wherein the method comprises:
   (a) providing an open-cell foam or fibrous substrate, wherein the open-cell foam or fibrous substrate comprises a polymer comprising heteroatom-containing moieties, wherein the polymer comprising heteroatom-containing moieties is selected from polyvinylpyridine, polyvinylpyrrolidone, polyvinyl alcohol, polyallylamine, polyethylene oxide, polyethylene imine, polyethylene sulfide and copolymers or blends thereof;
   (b) contacting the open-cell foam or fibrous substrate with nanoparticles of a first metal to provide a nanoparticle coated open-cell foam or fibrous substrate; and
   (c) contacting the nanoparticle coated open-cell foam or fibrous substrate with a solution comprising a salt of a second metal and a reducing agent to provide the metallized open-cell foam or fibrous substrate having a layer of the second metal on the nanoparticle coated open-cell foam or fibrous substrate.
6. The method according to any of items 1 to 5, wherein the first metal comprises Ag, Au, Pd, Pt, Rh, and Ru as well as alloys thereof.
7. The method according to any of items 1 to 6, wherein the second metal comprises Ag, Cu, Au, Pd, Pt, Rh, and Ru as well as alloys thereof.
8. Method according to claim 2, wherein the method comprises:
   (i) providing the open-cell foam or fibrous substrate, wherein the open-cell foam or fibrous substrate comprises melamine formaldehyde;
   (ii) contacting the open-cell foam or fibrous substrate with a solution comprising the polymer comprising heteroatom-containing moieties and a first solvent, wherein the polymer comprising heteroatom-containing moieties comprises polyvinylpyridine; and subsequently removing the first solvent; to provide the polymer coated open-cell foam or fibrous substrate;
   (iii) contacting the polymer coated open-cell foam or fibrous substrate with a suspension comprising nanoparticles of the first metal and the second solvent, wherein the first metal is selected from Ag, Au, Pd, Pt, Rh, and Ru as well as alloys thereof; and subsequently removing the second solvent, to provide the nanoparticle coated open-cell foam or fibrous substrate; and
   (iv) contacting the nanoparticle coated open-cell foam or fibrous substrate with the solution comprising the salt of the second metal, the reducing agent and a third solvent, wherein the second metal is selected from Ag, Cu, Au, Pd, Pt, Rh, and Ru as well as alloys thereof; and subsequently removing the third solvent, to provide the metallized open-cell foam or fibrous substrate having the layer of the second metal on the nanoparticle coated open-cell foam or fibrous substrate.
9. Method according to item 5, wherein the method comprises:
   (a) providing the open-cell foam or fibrous substrate, wherein the open-cell foam or fibrous substrate comprises polyvinylpyridine;
   (b) contacting the open-cell foam or fibrous substrate with a suspension comprising nanoparticles of the first metal and the second solvent, wherein the first metal is selected from Ag, Au, Pd, Pt, Rh, and Ru as well as alloys thereof; and subsequently removing the second solvent, to provide the nanoparticle coated open-cell foam or fibrous substrate; and
   (c) contacting the nanoparticle coated open-cell foam or fibrous substrate with the solution comprising the salt of the second metal, the reducing agent and a third solvent, wherein the second metal is selected from Ag, Cu, Au, Pd, Pt, Rh, and Ru as well as alloys thereof; and subsequently removing the third solvent, to provide the metallized open-cell foam or fibrous substrate having the layer of the second metal on the nanoparticle coated open-cell foam or fibrous substrate.
10. A metallized open-cell foam or fibrous substrate obtainable by the method according to any of items 1 to 9.
11. A lighting device comprising:
   a first conductive layer comprising a first metallized open-cell foam or metallized fibrous substrate according to item 10;
   a second conductive layer, which preferably comprises a second metallized open-cell foam or metallized fibrous substrate according to item 10;
   an insulating layer provided between the first conductive layer and the second conductive layer; and
   a lamp having a first electric contact which is connected to the first conductive layer and having a second electric contact which is connected to the second conductive layer.
12. A heating device comprising a metallized open-cell foam or metallized fibrous substrate according to item 10 and at least two electric contacts electrically connected with the metallized open-cell foam or metallized fibrous substrate.
13. Use of the metallized open-cell foam or fibrous substrate according to item 10 as a lighting device, a heating device, as a shield against electromagnetic irradiation, a filter, a catalyst, a substrate in analytical or preparative chromatography, or in a water separation device.
14. Use of the metallized open-cell foam or fibrous substrate according to item 10 as a thermal and/or sound insulation material, particularly in architectural applications, refrigeration devices or in vehicles.

The present invention is illustrated by the following non-limiting examples.

### Examples

Materials

AgNO₃ (p.A, Acros), alpha D(+) glucose (99+%, Acros), D/L tartaric acid anhydrous (>99%, Fluka), NaOH (≥98%, Sigma Aldrich), NH₄OH (24 wt %, Sigma Aldrich), ethanol (absolute, 99.9%, VWR), sodium citrate (98%, Acros), NaBH₄ (≥96%, Fluka), CuSO₄*5 H₂O (99+%, Acros), potassium sodium tartrate tetrahydrate (Acros), formaldehyde (37 wt %, Grüssing), PEG 400 (Fluka), pyridine (anhydrous, 99.8%, Aldrich), poly(4-vinylpyridine) (P4VP) (Mw=160 k, Aldrich), melamine formaldehyde open-cell foam (Basotect W), water (MilliQ plus freshly prepared) were used as received. MeOH (tech.) was distilled before use. Micrococcus luteus (M. Luteus) (Nr. 20300) and E. coli (Nr. 1077) were obtained from DSMZ Braunschweig.

Preparation of Silver Nanoparticle Suspension

8 nm sized silver nanoparticles (AgNP) were prepared with slight variations as reported in Liu C., Li B., Anal. Bioanal. Chem. 2011, 401, 229-235. In short: in a 1000 mL Schott flask 42 mg (0.247 mmol) AgNO₃ and 65 mg (0.252 mmol) sodium citrate were dissolved in 1000 mL water and stirred for 1 minute. Then 0.55 mg (0.015 mmol) NaBH₄ dissolved in 3 mL water was injected fast via a pipette to the solution and stirring was continued for 2 minutes. The resulting AgNP suspension was stored at 4° C. until use.

Surface Modification of Open-Cell Melamine Formaldehyde Foam with P4VP

In a 40 mL glass sample flask 100 mg P4VP was dissolved under stirring in 15 mL MeOH (6.6 mg/mL). A cube made of open-cell melamine formaldehyde foam (1) (10 mm*10 mm*10 mm) was immerged into the solution and was stirred for 1 minute. The open-cell foam was placed on a filter paper and the MeOH was pressed out of the open-cell foam until no further liquid was observed on fresh filter paper. Then the P4VP coated open-cell foam (2) was fully dried in vacuum at 80° C.

Loading of Melamine Formaldehyde Open-Cell Foam Surface with AgNP

In a 40 mL glass sample flask P4VP coated melamine formaldehyde open-cell foam was immersed in 10 mL of a 0.2 mM AgNP suspension. Then the solution was degassed twice at 1 mbar until the solution began to boil in order to degas the open-cell foam. The open-cell foam was treated on a shaker for 24 h. The AgNP suspension was removed and replaced by a fresh AgNP suspension. This was done four times until the open-cell foam surface was coated with nanoparticles resulting in a P4VP/AgNP coated open-cell foam (3).

Wet Chemical Deposition of Silver on P4VP/AgNP Coated Open-Cell Foam (3)

The Ag deposition suspension was prepared according to Lili L., Dan Y., Le W., Wie W., Journal of Applied Polymer Science 2012, 124, 1912-1918 using three different solutions:

1. Solution 1 was prepared by dissolving 5 g AgNO₃ in 100 mL of water.

2. Solution 2 was prepared by dissolving 0.05 g NaOH and 8.2 mL of ammonia solution (25 wt %) in 100 mL of water.

3. Solution 3 was prepared by dissolving 0.4 g D/L tartaric acid, 2.75 g alpha D(+) glucose and 10 mL of ethanol in 100 mL of water.

For homogeneous silver coating on an open-cell melamine formaldehyde foam 1 mL of solution 1 was mixed with 1 mL of solution 2 and combined with 2 mL of solution 3 in a 10 mL glass sample flask with a magnetic stir bar. P4VP/AgNP coated open-cell melamine formaldehyde foam (3) was placed in the flask, shaken, and carefully degassed and heated to 50° C. Samples were taken at different time intervals and rinsed with water. The wet open-cell foam samples were placed on filter papers for pre-drying followed by drying at 80° C. in vacuum.

Wet Chemical Deposition of Copper on AgNP Seeded Open-Cell Foams.

The copper deposition solution was prepared with modifications as reported previously by Hanna F., Hamid Z. A., Aal A. A. Materials Letters 2003, 58, 104-109. In short: In a 250 mL volumetric flask 2.5 g (10 mmol) CuSO₄*5 H₂O was dissolved in 100 mL water and 6.25 g (22 mmol) potassium sodium tartrate tetrahydrate was dissolved in the CuSO₄ solution. Then 2.5 g (62 mmol) NaOH, 500 mg PEG, 15 mg pyridine, and 7.5 mL of formaldehyde (37%) were dissolved in the solution. The flask was filled up to 250 mL and the solution was homogenized by shaking.

For homogeneous deposition of copper on P4VP/AgNP seeded open-cell foams (3), 20 mL of copper deposition solution was filled in a 40 mL glass sample flask with a magnetic stir bar and a venting hole in the lid. P4VP/AgNP coated open-cell melamine formaldehyde foam (3) was placed in the flask and the same procedure as in the silver deposition was used at a water bath temperature of 45° C. to get copper layers having varying thicknesses on the open-cell foam.

SEM Measurements

A LEO 1530 scanning electronic microscope (SEM) was used to obtain images of the surface of prepared silver and copper layers on open-cell melamine formaldehyde foams.

All samples were covered with 2 nm of platinum via a PVD sputtering device a sputter coater 208 HR from Cressington. The samples were glued on sample holders with water-based conductive carbon glue.

Resistivity Measurements

For resistivity measurements the metallized open-cell foams were pressed between two parallel 1 mm thick copper plates that were connected to a Keithley 2420 High-Current SourceMeter. Before measurement, the system resistivity of wires and plates were collected and subtracted from the measured values of the system and the open-cell foams. For every data point three samples were measured from all three sides and a mean value was calculated.

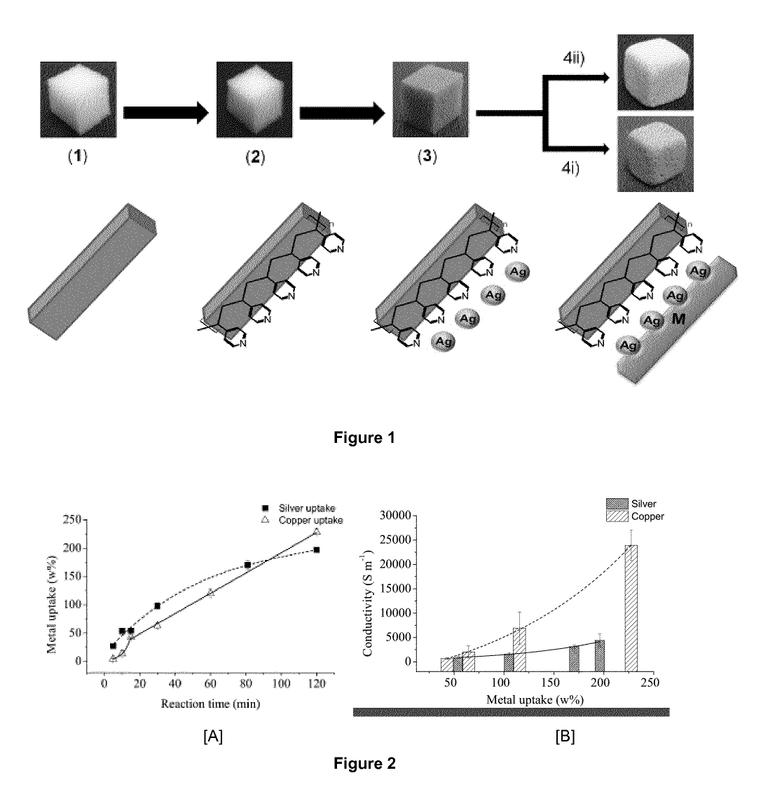

As can be seen from FIG. 2B, even if small amounts of silver and copper are deposited very low resistivities can be achieved. It was surprisingly found that a larger amount of copper had to be deposited compared to silver in order to achieve a measureable conductivity. After this limiting value had been exceeded the measured resistivity was much lower than that measured with the silver coated open-cell melamine formaldehyde foams. A longer reaction time for copper was needed due to an induction period.

Current Source Heating and Isolation Measurement

For heating up the silver coated open-cell melamine formaldehyde foam a DF-3010 lab power source was used. To measure the isolation behavior of silver covered, uncovered, and ground open-cell foam a MR 3001 K magnet stirrer with a hotplate from Heidolph was used. The hotplate was set to 100° C.

Three samples were investigated: a non-metallized open-cell melamine formaldehyde foam, a corresponding silver coated open-cell melamine formaldehyde foam, and a powder prepared from the silver coated open-cell melamine formaldehyde foam. The powder was prepared by grinding using a mortar. The weight of the three samples was the same.

The samples were placed on a hot plate which was set to 100° C. and were covered with a microscope slide. After 10 minutes, the temperature of the open-cell foams from both tests was measured with a SAT HotFind infrared camera with an emission coefficient set to 1.0 from ICOdata GmbH.

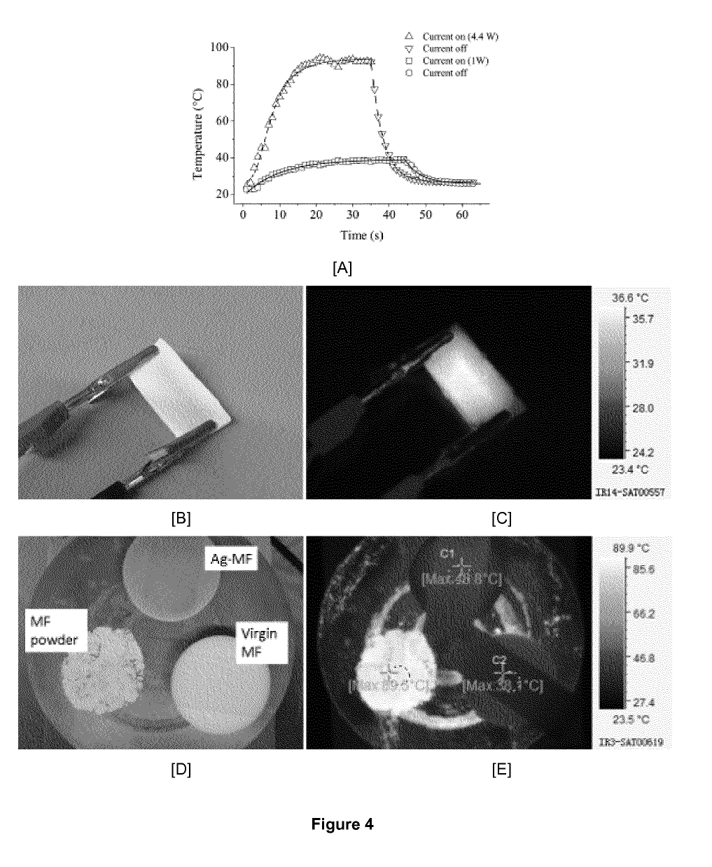

The experiments (FIGS. 4 (E,F)) showed that the microscope slide which was lying on the uncoated open-cell melamine formaldehyde foam had a temperature of about 38° C. Thus the foam provided good thermal insulation of the hot plate which had a temperature of about 100° C. The silver coated open-cell melamine formaldehyde foam had a temperature of about 49° C. and thus slightly reduced insulation properties. Compared to the powder of the silver coated open-cell melamine formaldehyde foam, the insulation properties were still excellent (temperature about 90° C.).

LED Lighting Device

To light up an LED, a sandwich consisting of a metallized open-cell melamine formaldehyde foam having a silver layer/uncoated open-cell melamine formaldehyde foam layer/metallized open-cell melamine formaldehyde foam having a silver layer was used. An array of 8 AA alkaline batteries (12V) was used to connect one open-cell foam positive and one negative. The wires of the LEDs (12V, Winger) were cut, so that one was insulated and only reached the lower level open-cell foam. The other wire was cut shorter and was insulated so that it can only be connected to the upper level open-cell foam. The same procedure was used to connect the batteries to the open-cell foams.

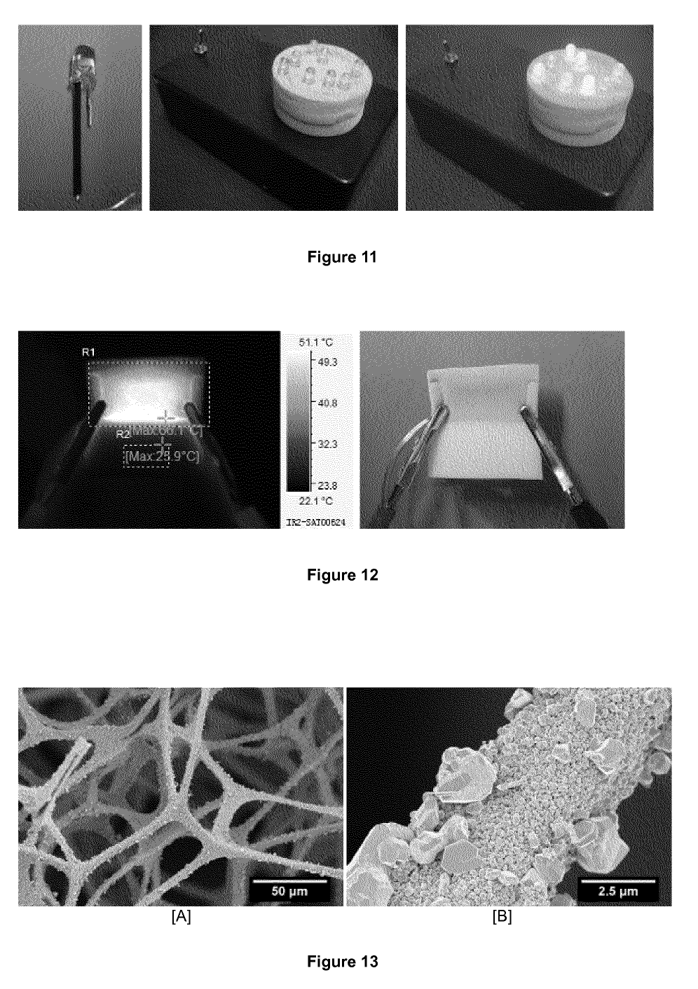

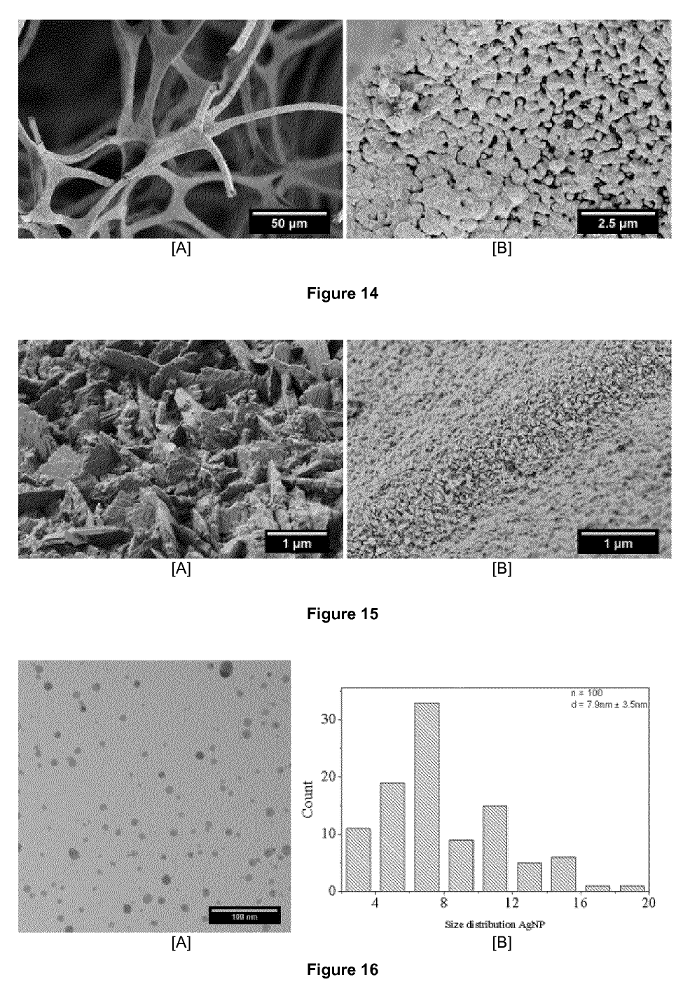

Due to the electrical conductivity of the metallized open-cell foams of the present invention, a lamp can be inserted at any position of the sandwich structure. The modified leads of the lamp close the electric circuit between the two metallized open-cell foam layers, so that the lamp glows. The lamps can therefore be positioned and repositioned at any desired position of the lighting device. The corresponding photographs are shown in FIG. 11.

Antibacterial Test

The concentration of the bacteria solution was determined via spectrophotometry. The optical density of 0.125 of the bacteria solution (E. coli) at 600 nm corresponded to 1.0×10¹⁰ CFU/mL. The bacterial solution was immediately diluted to 1.0×10⁸ CFU/mL and was the working bacterial solution for further tests. In the case of M. luteus, the optical density of 0.125 corresponded to 1.0×10⁶ CFU/mL. This bacteria solution was used as the working bacterial solution.

100 μL working bacterial solution was added to an agar plate and scratched out with a Drigalski spatula. The open-cell melamine formaldehyde foam coated with copper and silver with a size of 10 mm×10 mm×2 mm was put on the surface of the inoculated agar plate. The incubation ensued at 37° C. for 48 hours in the case of M. luteus and 18 hours in the case of E. coli.

After the incubation the open-cell melamine formaldehyde foam was raised carefully. This position was treated with an inoculation loop and a second agar plate was used for the scratching. The agar plate was incubated with the same parameters as described above.

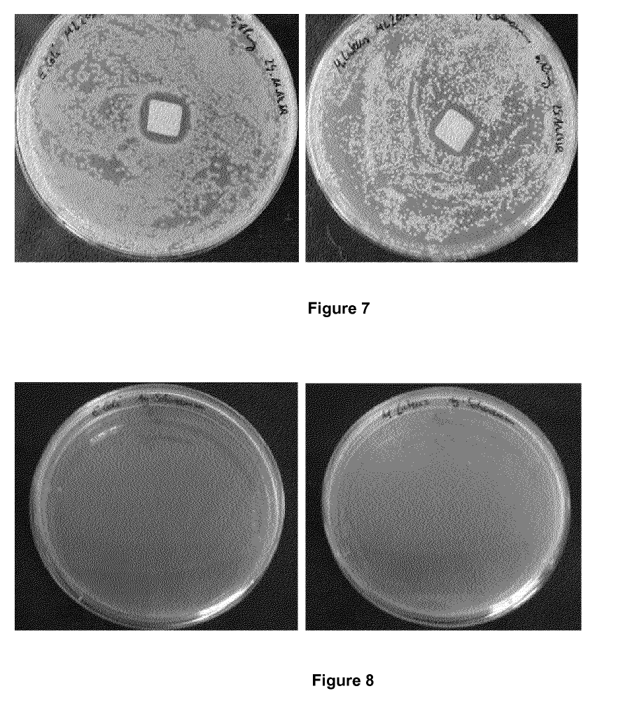

The silver coated open-cell melamine formaldehyde foams and uncoated open-cell melamine formaldehyde foams were subjected to a Kirby-Bauer test to evaluate their antimicrobial properties. The results are shown in FIGS. 7 to 10.

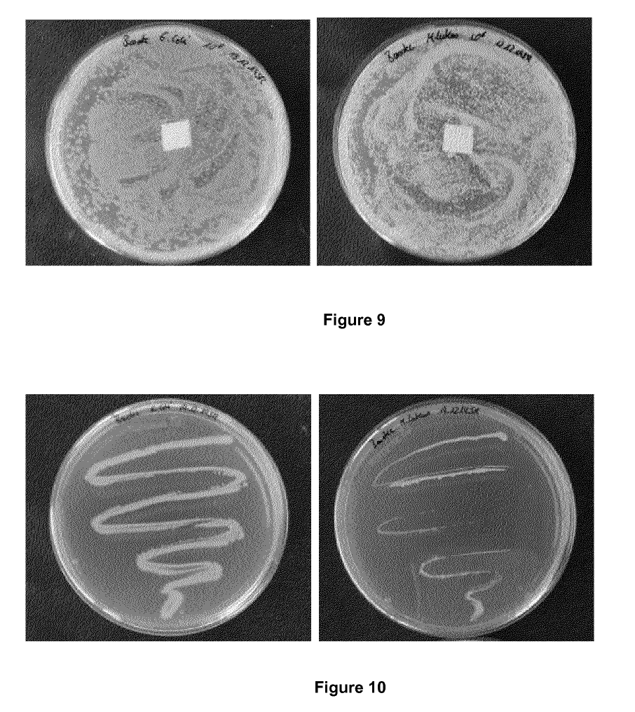

It can be seen that the silver coated open-cell melamine formaldehyde foam exhibited leaching and is thus antimicrobial. The uncoated open-cell foam did not have antimicrobial properties. After the incubation, samples were taken under the foams, the position was treated with an inoculation loop, and this was applied to a second agar plate. No bacteria grew along the path in the case of the silver coated open-cell melamine formaldehyde foam, whereas strong bacterial growth was observed along the path in the case of the uncoated open-cell melamine formaldehyde foam (FIGS. 9 and 10). This shows that the silver coated open-cell melamine formaldehyde foams have antibacterial properties. This is of particular interest because the open-cell melamine formaldehyde foams can be used as sound insulation and heat insulation in building or automotive applications. Furthermore, open-cell melamine formaldehyde foams are currently used as household sponges.

Contact Angle Measurement

The contact angle was measured at 20° C. using a drop shape analyzer DSA25S from Kruss. Drop size was controlled to 8 μL. For calculation, the Young Laplace method was used with ADVANCE software 1.1.0.2 from Kruss.

Wet Chemical Deposition of Copper and Silver on Open-Cell Melamine Formaldehyde Foams

Wet metallization of open-cell melamine formaldehyde foams was accomplished by successive pretreatment with P4VP followed by immobilization of AgNP, and finally by deposition of copper or silver layers (FIG. 1). For homogeneous metallization of silver and copper on melamine formaldehyde open-cell foams (1) pretreatment using a methanol solution of P4VP, followed by pretreatment with a dispersion of AgNP was essential. The successful deposition of AgNP on the open-cell foams caused a color change from orange to brown which remained even after intensive rinsing with water. Open-cell foam (3) became orange in color after deposition of copper and turned white-grey after deposition of silver.

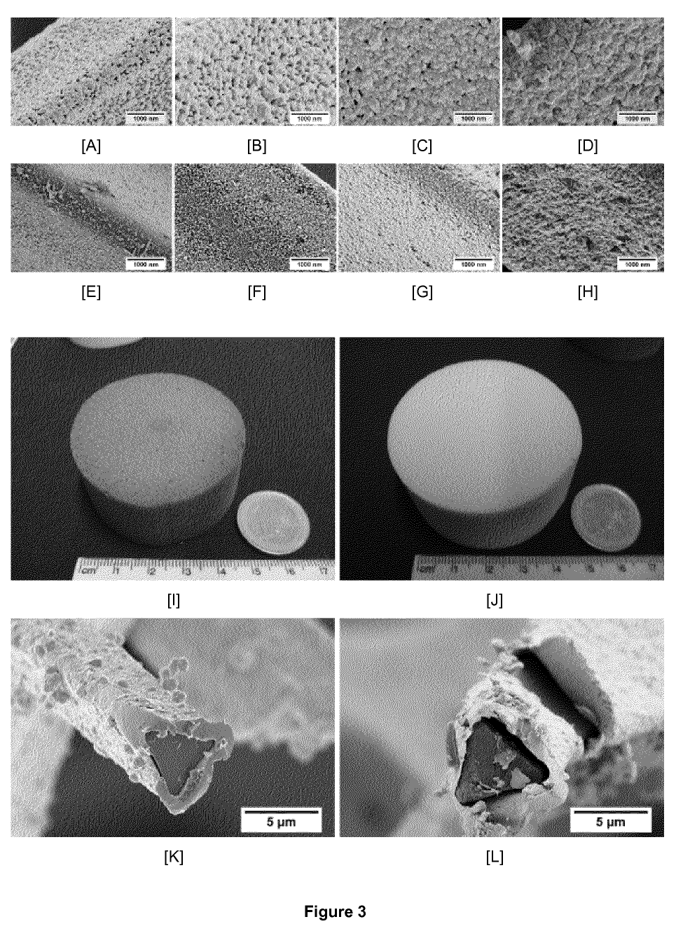

The progress of silver and copper deposition on open-cell foam (3) was monitored gravimetrically and correlated with gain in electrical conductivity of open-cell foam (4) (FIG. 2). The increase in electrical conductivity was accompanied by gradual change of the metal layer on the open-cell foam from an open granular morphology to a closed layer as observed by SEM (FIG. 3A-H). With longer reaction times continuous copper and silver layers were obtained as visualized by cross-sectional inspection with SEM (FIG. 3K, L).

A particular challenge in the deposition of copper was the liberation of hydrogen gas which can plug the pores of the open-cell foams and thereby prevented the penetration of the reaction mixture. This problem was overcome by reduction of the reaction rate using tartrate. A homogeneous continuous copper film even at a deposition thickness of 250 nm can be achieved by increasing the PEG surfactant concentration from 200 ppm to 2000 ppm.

In the case of copper, the quality of the metal layer could be improved by conducting the reaction under ambient conditions rather than under an argon atmosphere. The metallized open-cell foams showed relatively low densities of 31 mg cm⁻³ (4i) and 35 mg cm⁻³ (4ii).

With the formation of a continuous copper coating on the open-cell foam the resistivity decreased exponentially to a value of R=4 mΩ (2.4*10⁴ S/m) at an uptake of 228 wt %±6 wt % of copper. The final conductivity of (4i) was much higher than that of (4ii) (R=28 mΩ, 197 wt %+/−5 wt %, 4.4*10³ S/m). It is assumed that this might be due to crystalline defects in the silver layer due to the metallization process. Interestingly, the electrical conductivity of the open-cell melamine formaldehyde foams having a silver layer was high enough to operate LEDs in a sandwich set-up (FIG. 11). (4ii) (+200% silver) showed with dimensions of 25 mm*5 mm*50 mm and a regulated current of 5.2 A and 10.34 A significant electrical heating and cooling within seconds when the current was turned off (FIG. 4A). At a current of 5.2 A the voltage across (4ii) was measured with 0.195 V (1 W). The open-cell foam reached a temperature of 37° C. under these conditions in 19 s as visualized by IR-camera (FIG. 4C). Simultaneously, the silver coated open-cell melamine formaldehyde foams also displayed heat insulating properties similar to the uncoated open-cell melamine formaldehyde foams (FIGS. 4D, 4E). In contrast, a ground sample of the silver coated open-cell foam showed no heat insulation (FIG. 4E).

Superhydrophobic Properties of a Silver or Copper Coated Open-Cell Melamine Formaldehyde Foam

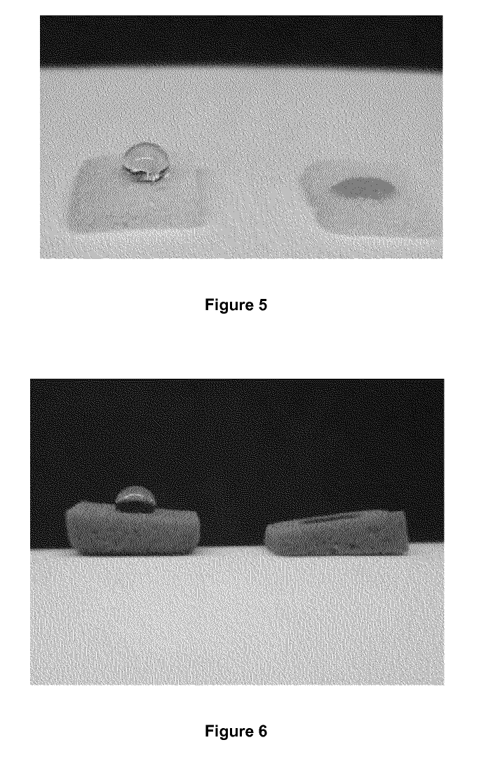

It was surprisingly found that the silver or copper coated open-cell melamine formaldehyde foams had superhydrophobic properties. Corresponding photographs are shown in FIGS. 5 and 6.

In contrast to uncoated open-cell melamine formaldehyde foam (1) the open-cell melamine formaldehyde foams (4i) and (4ii) showed high contact angles against water (FIG. 5) but sliding of water droplets when the surfaces of the open-cell melamine formaldehyde foams were tilted. The contact angle of open-cell melamine formaldehyde foam (4i) exhibited a similar contact angle (151°±3°) to open-cell melamine formaldehyde foam (4ii) (152°±4°). Superhydrophobic metal surfaces were reported previously (Liu, K.; Jiang, L. Nanoscale 2011, 3, 825) and were attributed to fractal metal surfaces, which could also apply to open-cell melamine formaldehyde foams (4). Very interesting and to the best of our knowledge not yet reported was the observation that water droplets injected in the open-cell melamine formaldehyde foams were ejected from the open-cell melamine formaldehyde foams rather rapidly. In contrast, (4i) and (4ii) were wetted by oil which makes the open-cell melamine formaldehyde foams promising membranes for oil/water separation.

The uncoated open-cell melamine formaldehyde foam did not possess these properties: it sucked up both oil and water.

Use of the Metallized Open-Cell Foams as Heating Elements

The properties of a silver coated open-cell melamine formaldehyde foam were investigated when high electric currents were passed through. A 50 mm×20 mm×5 mm foam was contacted by two alligator clips and currents of 5.2 A and 10.44 A were applied. The heating of the foam was visualized using an IR camera. The corresponding photographs and thermographs are shown in FIGS. 4C, 4D and 12.

In a direct comparison, it can be seen that the uncoated open-cell melamine formaldehyde foam did not heat up. In contrast thereto, the silver coated open-cell melamine formaldehyde foam heated up to a temperature of about 66° C. This shows the potential for combining heat insulation and heating function in a single material.

The measurements showed that the silver coated open-cell melamine formaldehyde foam had a very low resistivity. The voltage drop at a current of 5.2 A was just 0.195 V. Under these conditions the silver coated open-cell melamine formaldehyde foam was heated to about 37° C. during 30 s. At a current of 10.34 A the silver coated open-cell melamine formaldehyde foam heated to about 90° C. during 25 s and reached room temperature again after about 10 s. The metallized open-cell melamine formaldehyde foam is thus particularly suitable as a heating element which can emit heat very quickly due to its low mass and can also quickly terminate the heat emission after the flow of electricity has been stopped.

### CONCLUSIONS

With the present method, it was possible to generate homogeneous metal layers with a thickness of less than 500 nm on open-cell foams. These foams showed very good conductivities of up to 2.4*10⁴ S/m, antibacterial and hydrophobic properties, thermal insulation, and the ability of small samples to resist currents up to 10 A. They are also an outstanding material for electrodes, heating elements, high temperature, antistatic, and antibacterial filters.

Homogeneous deposition of first metal nanoparticles on open-cell melamine formaldehyde foams allowed the deposition of thick and persistent second metal layers. The metallized open-cell foams showed excellent electrical conductivities of up to σ=2.4*10⁴ S m⁻¹ and tolerated currents of up to 10 A. Without current flow the metallized open-cell foams were thermal insulators which make them of particular interest as thermal devices. Large contact angles against water of the metallized open-cell foams were an unexpected feature, which is of interest for oil/water separation and the prevention of fouling. However, it should be stressed, that sliding of water on the surface of the metallized open-cell foams was found which means that the three phase contact line showed inhomogeneities (Chen, W.; Fadeev, A. Y.; Hsieh, A. C.; Öner, D.; Youngblood, J.; McCarthy, T. J. Langmuir 1999, 15, 3395). Nevertheless, water droplets injected in the metallized open-cell foams were rapidly ejected which should prevent unwanted water-uptake and follow up reactions of such metallized open-cell foams.

Copper Plated Polyurethane Foams

Four pieces of polyurethane foams (20×20×10 mm) were immersed into a solution containing 10 mg/L polyethyleneimine (PEI) in water. The foams were compressed with an stamp and pressure was released to remove all air out of the foams. After this the foams were washed with water to remove remaining PEI.

The wet, washed samples were covered with silver nanoparticles (AgNP) as described above with respect to the melamine foams.

After this the foams were coated in 300 mL copper plating bath like the melamine foams to obtain a copper uptake of 113%. The density of the foams was determined to be 55 mg cm⁻³ after electroless plating. The conductivity was 4940 S m⁻¹.

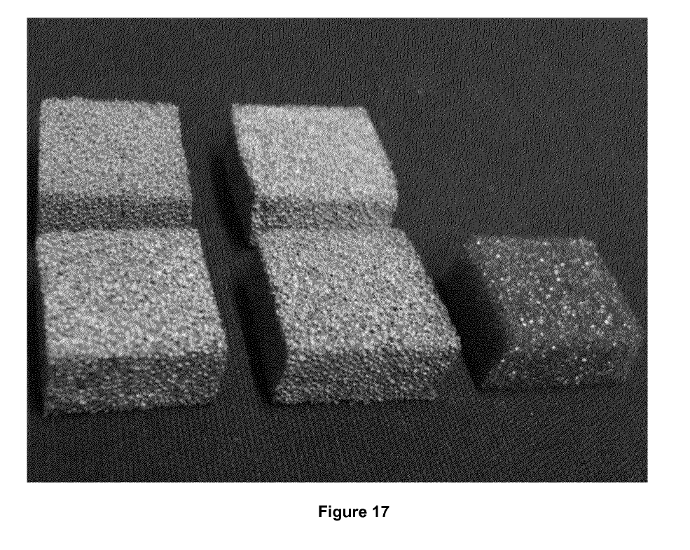

FIG. 17 shows the copper plated polyurethane foams (left) and an untreated foam (right).

1. Method for preparing a metallized open-cell foam or fibrous substrate the method comprising:
providing an open-cell foam or fibrous substrate, wherein the open-cell foam or fibrous substrate contains a polymer comprising heteroatom-containing moieties within the bulk of the open-cell foam or fibrous substrate or as a coating on the open-cell foam or fibrous substrate, wherein the polymer comprising heteroatom-containing moieties is selected from polyvinylpyridine, polyvinylpyrrolidone, polyvinyl alcohol, polyallylamine, polyethylene oxide, polyethylene imine, polyethylene sulfide and copolymers or blends thereof;
contacting the open-cell foam or fibrous substrate with nanoparticles of a first metal to provide a nanoparticle coated open-cell foam or fibrous substrate; and
contacting the nanoparticle coated open-cell foam or fibrous substrate with a second solution comprising a salt of a second metal and a reducing agent to provide the metallized open-cell foam or fibrous substrate having a layer of the second metal on the nanoparticle coated open-cell foam or fibrous substrate.

2. The method according to claim 1, wherein the step of providing an open-cell foam or fibrous substrate comprises
contacting an open-cell foam or fibrous substrate with a polymer comprising heteroatom-containing moieties, wherein the polymer comprising heteroatom-containing moieties is selected from polyvinylpyridine, polyvinylpyrrolidone, polyvinyl alcohol, polyallylamine, polyethylene oxide, polyethylene imine, polyethylene sulfide and copolymers or blends thereof, to provide a polymer coated open-cell foam or fibrous substrate.

3. The method according to claim 2, wherein the open-cell foam or fibrous substrate comprises polymer material selected from melamine formaldehyde resin, polyurethane, polyamide, polyimide, polyester polyurethane, polyether polyurethane, and combinations thereof.

4. The method according to claim 1, wherein the polymer comprising heteroatom-containing moieties comprises polyvinylpyridine.

5. A method for preparing a metallized open-cell foam or fibrous substrate the method comprising:
providing an open-cell foam or fibrous substrate, wherein the open-cell foam or fibrous substrate comprises a polymer comprising heteroatom-containing moieties selected from polyvinylpyridine, polyvinylpyrrolidone, polyvinyl alcohol, polyallylamine, polyethylene oxide, polyethylene imine, polyethylene sulfide and copolymers or blends thereof;
contacting the open-cell foam or fibrous substrate with nanoparticles of a first metal to provide a nanoparticle coated open-cell foam or fibrous substrate; and
contacting the nanoparticle coated open-cell foam or fibrous substrate with a second solution comprising a salt of a second metal and a reducing agent to provide the metallized open-cell foam or fibrous substrate having a layer of the second metal on the nanoparticle coated open-cell foam or fibrous substrate.

6. The method according to claim 1, wherein the first metal comprises Ag, Au, Pd, Pt, Rh, Ru, or alloys thereof.

7. The method according to claim 1, wherein the second metal comprises Ag, Cu, Au, Pd, Pt, Rh, Ru, or alloys thereof.

8. The method according to claim 2,
wherein the open-cell foam or fibrous substrate comprises melamine formaldehyde, wherein the step of contacting the open-cell foam or fibrous substrate with a polymer comprises contacting the open-cell foam or fibrous substrate with a first solution comprising the polymer comprising heteroatom-containing moieties and a first solvent,
wherein the polymer comprising heteroatom-containing moieties comprises polyvinylpyridine,
wherein the polymer coated open-cell foam or fibrous substrate is contacted with a suspension comprising nanoparticles of the first metal and a second solvent,
wherein the first metal is selected from Ag, Au, Pd, Pt, Rh, Ru, and alloys thereof
wherein the nanoparticle coated open-cell foam or fibrous substrate is contacted with a second solution further comprising a third solvent, and
wherein the second metal is selected from Ag, Cu, Au, Pd, Pt, Rh, Ru, and alloys thereof.

9. A method according to claim 5, wherein the open-cell foam or fibrous substrate comprises polyvinylpyridine,
wherein the open-cell foam or fibrous substrate is contacted with a suspension comprising nanoparticles of the first metal and a second solvent,
wherein the first metal is selected from Ag, Au, Pd, Pt, Rh, Ru, and alloys thereof
wherein the nanoparticle coated open-cell foam or fibrous substrate is contacted with the second solution further comprising a third solvent,
wherein the second metal is selected from Ag, Cu, Au, Pd, Pt, Rh, Ru, and alloys thereof.

10. A metallized open-cell foam or fibrous substrate produced according to the method of claim 1.

11. A lighting device comprising:
at least one conductive layer comprising a first metallized open-cell foam or metallized fibrous substrate according to claim 10;
a second conductive layer;
an insulating layer between the at least one conductive layer and the second conductive layer; and
a lamp comprising a first electric contact electrically connected to the first conductive layer and a second electric contact electrically connected to the second conductive layer.

12. A heating device comprising a metallized open-cell foam or metallized fibrous substrate according to claim 10 and at least two electric contacts electrically connected with the metallized open-cell foam or metallized fibrous substrate.

13. (canceled)

14. (canceled)

15. A lighting device
a first and a second conductive layer comprising first metallized open-cell foam or metallized fibrous substrates according to claim 10;
an insulating layer provided between the first conductive layer and the second conductive layer; and
a lamp comprising a first electric contact electrically connected to the first conductive layer and having a second electric contact electrically connected to the second conductive layer.

16. (canceled)

17. The method according to claim 2, wherein the open-cell foam or fibrous substrate comprises melamine formaldehyde.

18. The method according to claim 8 further comprising removing the first solvent after contacting the open-cell foam or fibrous substrate with the first solution to obtain the polymer coated open-cell foam or fibrous substrate.

19. The method according to claim 8 further comprising removing the third solvent after contacting the polymer coated open-cell foam or fibrous substrate with the second solution to obtain the metallized open-cell foam or fibrous substrate having the layer of the second metal on the nanoparticle coated open-cell foam or fibrous substrate.
